(19) United States
(12) Patent Application Publication
(10) Pub. No.: US 2018/0008810 A1
(43) Pub. Date: Jan. 11, 2018

(54) VALVE DEVICE, REMOVING TOOL AND MOUNTING TOOL

(71) Applicant: CENDRES+MÉTAUX SA, Bienne (CH)

(72) Inventors: Hans MESSERLI, Kriegstetten (CH); Adrian AUDERSET, Nidau (CH); Daniel SCHUTZ, Aarwangen (CH)

(21) Appl. No.: 15/543,588

(22) PCT Filed: Feb. 20, 2015
(86) PCT No.: PCT/EP2015/053637

§ 371 Date: Jul. 14, 2017

(51) Int. Cl. A61M 39/02; A61M 39/24

(52) U.S. Cl. CPC A61M 39/0247; A61M 2039/027; A61M 2039/2493

(57) ABSTRACT

A valve device suitable for detachably fixing to an access port and includes a fixation clip which has at least a first and second fixation member each of which have one or more rims each having a surface which veers in a direction towards the valve device. There is further provided a removing tool for removing the valve device from an access port. There is further provided a mounting tool for mounting a valve device in an access port. An assembly having a mounting tool and a valve device is also provided.

### FIELD OF THE INVENTION

The present invention relates to a valve device a corresponding removing tool which can be used to remove the valve device from an access port and a corresponding mounting tool which can be used to mount a valve device on an access port. There is further provided a valve device with which said tools can cooperate.

### DESCRIPTION OF RELATED ART

Access ports, such as those described in WO2011/150978, contain valve devices which typically require replacement on a regular basis. Removing these valve devices is difficult and requires high dexterity. Removing these valve devices can also be risky due to blood loss or air suction into the patient's blood system. Additionally, positioning a new replacement valve device onto the access port is also difficult and requires high dexterity.

It is an aim of the present invention to mitigate or obviate at least some of the problems mentioned above.

### BRIEF SUMMARY OF THE INVENTION

According to a first aspect of the present invention there is provided a valve device suitable for detachably fixing to an access port, the valve device comprising, one or more valves which are selectively operable to be in an open or closed configuration; a clip which comprises at least a first and second fixation member, wherein, the fixation members are arrangeable to mechanically cooperate with the access port to detachably fix the valve device to the access port, wherein each of the fixation members comprises one or more rims which each have a surface which veers in a direction towards the valve device, so that a tool can mechanically cooperate with said surfaces of the rims to remove the valve device from the access port.

Preferably each of the one or more rims is defined by a projection which is provided on the fixation member. The projection may be an elongate projection.

Preferably the fixation members are configured such that they can snap-fit onto the access port to detachably fix the valve device to the access port.

The access port may be an access port which is described in WO2011/150978.

Each fixation member may comprise two rims, each provided at opposite sides of each fixation member, wherein each rim has a surface which veers towards the valve device.

The valve device may comprise a housing which houses the one or more valves, and wherein each fixation member has a first end which is secured to the housing and a second, free end, and, wherein each rim is configured so that it has a surface which, in the direction from the first end of the fixation member to the second end of the fixation member, veers in a direction towards the housing.

Each rim may be configured so that said surface is substantially perpendicular to the direction in which the projection projects from the fixation member which comprises that rim.

Each of the fixation members may comprise a projection at one end which can be received into a recess provided in the access port, to detachably fix the valve device to the access port.

The thickness of each rim may increase along the fixation member. Preferably the thickness of each rim is a maximum at the end of the fixation member which comprises the projection. So in other words, preferably the thickness of the rim increases from the first end of the fixation member to the second end of the fixation member.

Each fixation member may comprise flexible material which allows the fixation member to flex. Preferably the flexible material will allow the fixation member to flex in a direction away from the housing. The flexible material of the fixation members will enable the valve device to snap-fit onto the access port, to detachably fix the valve device to the access port. Preferably the fixation members will flex as the projections on the fixation members are forced over a retaining lip on the access port; when the projections on the fixation members are forced over a retaining lip on the access port they will be received into corresponding recesses provided on the access port, and will thus recoil from their flexed position thereby snap-fitting the valve device to the access port.

It will be understood that the projections do not need to be received into corresponding recesses to detachably fix the valve device to the access port; the valve device may be snap fitted to the access port simply by the projections on the fixation members sliding over the retaining lip on the access port. In this case the retaining lip on the access port will retain the valve device on the access port and the projections. Likewise it will be understood that a retaining lip on the access port is also not required to detachably fix the valve device to the access port; the valve device may be snap fitted to the access port simply by the projections on the fixation members being received into corresponding recesses provided on the access port.

Each of the fixation members may comprises a ledge which can mechanically cooperate with a clip of a tool so that the tool can hold the valve device at the fixation members. Preferably each of the fixation members comprises a ledge which can mechanically cooperate with a clip of a tool so that the tool can hold the valve device at the fixation members so that the valve device is fixed in the tool. The ledge may mechanically cooperate with a clip of a tool by friction. Preferably the ledge on each fixation member comprises a projection which will enable the fixation members snap-fit into the clip of a tool so that the tool can hold the valve device at the fixation members. Preferably the projection provided on each ledge is an elongate projection. Preferably each ledge is configured such that it can mechanically cooperate with a clip of a tool only when the respective fixation member has been flexed in a direction away from the housing.

The fixation members may be arranged symmetrically on the valve device. Preferably the first and second fixation member are arranged on opposite sides of the valve device. The first and second fixation members are preferably attached at opposite sides of the housing. Preferably the valve device comprises two valves allowing inflow and outflow of fluids.

The valve device may be adapted to be detachably fixed to an access port for removing and/or returning of fluids to a patient, and wherein a first valve of the valve device is connected to a first conduit which can carry fluid to the patient and a second valve of the valve device is connected to a second conduit which can carry fluid from the patient. When a respective valve is in its open configuration it allows fluid communication between the access port and the patient and when the respective valve is in its closed configuration it blocks fluid communication between the access port and the patient. Preferably the valve device comprises two valves and one valve cooperates with a conduit which carries fluid to the patient and the other valve cooperates with a conduit which carries fluid from the patient.

According to a further aspect of the present invention there is provided a removing tool for removing the valve device according to any one of valve devices mentioned above which is detachably fixed to an access port, the removing tool comprising: at least two elements each of which can be arranged to abut with said surface of a rim provided on a respective fixation member of the valve device, wherein the elements are fixed in the removing tool so that each of the elements is moved along a respective surface of the rim of a respective fixation member as the removing tool is advanced towards the valve device, so that the fixation members are moved apart and the valve device becomes ultimately detached from the access port; a fixation clip arranged for holding the valve device after it has become detached from the access port, so that the valve device is fixed to the removing tool after the valve device has been detached from the access port by the elements. Preferably, the removing tool is designed in a way that it cannot release the valve device once it has been fixed in the holding cavity of the removing tool An advantage of this configuration of the removing tool is that the removed valve cannot be used again in the access port or any other access port.

Preferably the fixation clip is configured so that it can hold the valve device at the fixation members.

The fixation clip may be positioned so that moving the fixation members apart using the elements by advancing the removing tool towards the valve device, moves the fixation members to clip into the fixation clip, so that the fixation clip holds the valve device at its fixation members.

The fixation clip preferably comprises a first and second member which are arranged opposite one another.

Each of the first and second members may comprise a projection which can mechanically cooperate with a ledge provided on a respective fixation member of the valve device, to hold the valve device. The projection may mechanically cooperate with a respective ledge by friction. The projection may be c-shaped. This will allow the fixation member to snap-fit into each of the first and second members. For example, if the ledge on each fixation member of the valve device comprises a projection, this projection may snap into the c-shaped projection, so that the valve device is held in the fixation clip.

The distance between the first and second members may be such that moving the fixation members apart using the elements by advancing the removing tool towards the valve device, moves a first fixation member so that the projection on the first member mechanically cooperates with a ledge provided on the first fixation member, and, moves the second fixation member so that the projection on the second member mechanically cooperates with a ledge provided on the second fixation member, so that the fixation clip holds the valve device at its fixation members.

Each of the first and second members may comprise a tapered member and said projection which is provided at an end of the tapered member. Each tapered member may be attached at its widest end to the removing tool, and the narrowest end of the tapered member may be a free end, and the projection may be located at the narrowest end of the tapered member.

The at least two elements may be arranged opposite one another on the removing tool.

The distance between outer surfaces of the elements may be equal to the shortest distance between the surface of a rim provided on the first fixation member and the surface of a rim provided on the second fixation member of the valve device.

Each of the two elements may be a projection. Preferably each of the two elements is a projection which projects from a body of the removing tool. Each of the at least two elements may be a tapered projection. Preferably the longer side of each tapered projection can abut said surface of the rim on a respective fixation member.

The removing tool may further comprise reinforcing members which provide structural support to each of the elements.

The removing tool further comprises a handle portion. The reinforcing members may attach each of the elements to the handle portion.

The removing tool may further comprise at least two projecting platforms which can frictionally engage a valve device which is held by the fixation clip. The projecting platforms will ensure that the valve device is fixed more securely to the removing tool. Additionally the projecting platforms will facilitate positioning of a valve device in a predetermined position in the removing tool when the valve device is being removed from the access port.

Preferably each of the projecting platforms further comprises nodules which ensure that the valve device is fixed more securely to the removing tool. The nodules increase friction between a respective projecting platform and a valve device which is held by the fixation clip.

Preferably the removing tool is defined by a single molded structure.

Preferably the removing tool comprises a signal colored material (e.g. red). Preferably the removing tool is composed of a signal colored material (red or other). The signal colored material can be useful in indicating that the removing tool is intended to be used only once and thrown away immediately after use.

The removing tool may comprise two pairs of elements the members of each pair been arranged opposite one another on the removing tool. The elements may be arranged such that the distance between outer surfaces of one pair of elements is equal to the shortest distance between said surfaces of rims provided on one side of a pair of fixation members of the valve device, and the distance between outer surfaces of the other pair of elements is equal to the shortest distance between said surfaces of rims provided on an opposite side of said pair of fixation members of the valve device. Thus, four elements will be provided, a first element which can be arranged to abut said surface of a rim provided at one side of first fixation member, and, a second element which can be arranged to abut said surface of a rim provided at the opposite side of the first fixation member; and a third element which can be arranged to abut said surface of a rim provided at one side of a second fixation member, and, a fourth element which can be arranged to abut said surface of a rim provided at the opposite side of the second fixation member. The first and third element will define the first pair of elements and the second and fourth element will define the second pair of elements.

According to a further aspect of the present invention there is provided a mounting tool for mounting a valve device according to any one of the above-mentioned valve devices in an access port, the mounting tool comprising: a cavity defined in a body of the mounting tool, suitable for receiving a valve device; and a holding clip into which the fixation members of a valve device which is positioned in the cavity can clip only when the fixation members are flexed apart, so as to hold the valve device in the cavity only when the fixation members are flexed apart.

A valve device to be mounted on an access port may be positioned into the cavity of the mounting tool before use. The valve device is brought to the position on the access port where it is to be detachably fixed. The fixation members are preferably designed to snap-fit onto the access port, to detachably fix the valve device to the access port; fixation members will flex as the projections on the fixation members are forced over a retaining lip on the access port. When the projections on the fixation members are forced over a retaining lip on the access port they will be received into corresponding recesses provided on the access port, and will thus recoil from their flexed position thereby snap-fitting the valve device to the access port. Accordingly, as the user pushes the valve device into position on the access port the fixation members will be flexed apart. Once the fixation members have been flexed apart they will clip into the holding clip of the mounting tool. At this point the holding clip will prevent the valve device from being removed from the tool until the fixation members recoil. Once the valve device has been pushed into position on the access port the fixation members will recoil (so that they are no longer flexed apart) as the projections on the fixation members are received into respective recesses on the access port. Only when the fixation members recoil and are correctly placed in the respective recesses on the access port such that the valve device is properly fixed to the access port, they will become released from the holding clip so that the valve device is no longer held in the cavity of the mounting tool. Thus, the mounting tool can be removed safely, leaving the valve device detachably fixed to the access port.

Preferably the holding clip is configured to release the fixation members when fixation members recoil from their flexed position.

The holding clip preferably comprises a first and second member located as opposite sides of the cavity.

Preferably the first and second members are configured so they can each grip a fixation member of the valve device, so that the fixation members are clipped into the holding clip. When each of the first and second members grip a fixation member of the valve device the fixation members are considered to be clipped into the holding clip.

Preferably the distance between the first and second members is such that the fixation members clip into the holding clip only when the fixation members are flexed apart. Preferably the distance between the first and second members is such that, said projection on the first member can grip a ledge provided on the first fixation when the first fixation member is flexed as the valve device is pushed into a position on an access port where the valve device is to be detachably fixed, and, said projection on the second member can grip a ledge provided on the second fixation member when the second fixation member is flexed as the valve device is pushed into a position on an access port where the valve device is to be detachably fixed, so that the holding clip fixedly holds the valve device when the fixation members are flexed apart as the valve device is being pushed into a position on an access port where it is to be detachably fixed.

When the valve device has been moved into position in the access port, the fixation members will retract from their flexed position such that the valve device becomes detachably fixed to the access port. This will ensure that the fixation members will be released from (i.e. unclipped) the holding clip when the fixation members recoil such that the valve device is released from the mounting tool. The fixation members will recoil when the projections on the fixation members have been slid over a retaining lip on the access port and have been received into corresponding recesses provided on the access port, and will thus recoil from their flexed position thereby snap-fitting the valve device to the access port. An advantage of this configuration is that as long as the valve device is not correctly positioned on the access port or not correctly fixed to it (for example only one fixation member has been clipped in a respective recess on just one side of the access port) the projections of the holding clip will not release the valve device from the mounting tool.

Each of the first and second members may comprise a tapered member and said projection which is provided at an end of the tapered member. Each tapered member may be attached at its widest end to the mounting tool, and the narrowest end of the tapered member is a free end, and the projection is located at the narrowest end of the tapered member.

The mounting tool may further comprise one or more projecting platforms which hold a valve device in the cavity. Preferably the one or more projecting platforms hold a valve device in a predetermined position in the cavity.

Preferably the one or more projecting platforms comprise one or more nodules which increase friction between a valve device in the cavity and each projecting platform so that the valve device is held more securely in the cavity.

Preferably the mounting tool is defined by a single molded structure.

The mounting tool can comprise transparent material or is composed of transparent material. Preferably, the mounting tool is composed of a colored material, the color possibly corresponding to a code such as “attention, not to be reused!”.

According to a further aspect of the present invention there is provided an assembly comprising a mounting tool according to any one of the above-mentioned mounting tools and valve device according to any one of the above mentioned valve devices, which is held in the cavity of the mounting tool.

### BRIEF DESCRIPTION OF THE DRAWINGS

The invention will now be described, by way of example only, with reference to the following figures in which:

FIG. 1 shows a perspective view of a valve device according to an aspect of the present invention;

FIG. 2 shows a perspective view of a removing tool according to a further aspect of the present invention;

FIG. 3 shows a perspective view of a removing tool of FIG. 2 with the valve device of FIG. 1 fixed to the removing tool after having been detached from an access port;

FIG. 4 shows a perspective view of a mounting tool according to a further aspect of the present invention;

FIG. 5 shows a perspective view of the mounting tool of FIG. 4 with the valve device of FIG. 1 held in the cavity of the mounting tool.

### DETAILED DESCRIPTION OF POSSIBLE EMBODIMENTS OF THE INVENTION

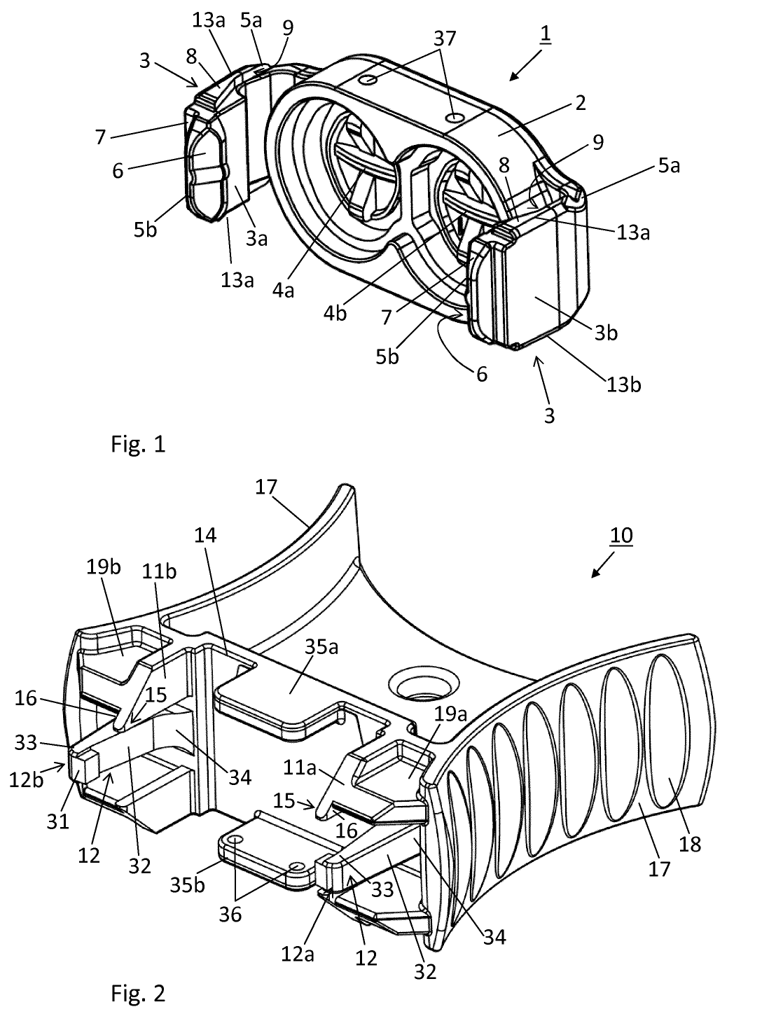

FIG. 1 shows a perspective view of a valve device 1 according to a first aspect of the present invention. The valve device 1 is suitable to be fixed detachably to an access port such as the access port described in WO2011/150978; the access port disclosed in WO2011/150978 is used for removing and/or returning of fluids to a patient.

The valve device 1 comprises two valves 4a, 4b (represented as slit valves in FIG. 1) which are selectively operable to be both in an open or closed configuration. The two valves 4a, 4b are housed in a housing 2 of the valve device 1. In use a first valve 4a of the valve device may be connected to a first conduit which can carry fluid to the patient and a second valve 4b of the valve device 1 may be connected to a second conduit which can carry fluid from the patient. When the valves 4a, 4b are in the open configuration they will allow fluid communication between the access port and the patient. When the valves 4a, 4b are in its closed configuration they block fluid communication between the access port and the patient and can further prevent air suction (from outside in).

The valve device 1 comprises a clip 3 which comprises a first and second fixation member 3a, 3b. Each fixation member 3a, 3b has a first end 5a which is secured to the housing 2 and a second, free end 5b. The fixation members 3a, 3b are arranged symmetrically on the valve device 1; the first and second fixation member 3a, 3b are arranged at opposite sides the housing 2.

The fixation members 3a, 3b are arrangeable to mechanically cooperate with the access port to detachably fix the valve device 1 to the access port. In this example, the fixation members 3a, 3b are configured such that they can snap-fit onto the access port to detachably fix the valve device to the access port; each of the fixation members 3a, 3b comprises a projection 6 which is located at its second end 5b which can be received into a corresponding recess provided in the access port; each fixation members 3a, 3b comprises flexible material which allows each of the fixation members 3a, 3b to flex in a direction away from the housing 2. The flexible material of the fixation members 3a, 3b and the projections 6 will enable the valve device to snap-fit onto the access port, to detachably fix the valve device to the access port. To detectably fix the valve device 1 the fixation members 3a, 3b will flex to allow the projections 6 to slide over a retaining lip provided on the access port, and once the projections 6 have been slid over the retaining lip the flexed fixation members 3a, 3b will recoil so that the projections 6 are received into corresponding recesses on the access port, so that the valve device 1 is snap-fitted to detachably fix to the access port.

Each of the fixation members 3a, 3b comprises a ledge 7 with which a clip of a tool can mechanically cooperate so that tool can hold the valve device at the fixation members 3a, 3b. As will be discussed in more detail later, members of the clip of the tool may mechanically cooperate with a ledge 7 on each respective fixation member 3a, 3b, by abutting the ledge 7;such as to hold the valve device 1 at the fixation members 3a, 3b. Each ledge 7 is positioned such that it can mechanically cooperate with a clip member of a tool only when the respective fixation member 3a, 3b has been flexed in a direction away from the housing 2. In an alternatively embodiment an elongate projection may be provided on the ledge 7 of each fixation member 3a, 3b so as to enable the fixation members 3a, 3b snap-fit into a clip of a tool, so that the clip of the tool can hold the valve device 1 at the fixation members 3a, 3b.

Each of the fixation members 3a, 3b comprises a rim 8 which has a surface 9 which, in the direction from a first end 5a of the fixation member 3a, 3b to a second end 5b of the fixation member 3a, 3b, veers in a direction towards the housing 2. The rim 8 on each fixation member 3a, 3b is configured so that said surface 9 is substantially perpendicular to the direction in which the projection 6 on the respective fixation member 3a, 3bprojects. Each rim 8 is defined by an elongate projection which is provided on the respective fixation member 3a, 3b. The thickness of each rim 8 increases along the fixation member 3a, 3b, in the direction from the first end 5a of the fixation member 3a, 3b to the second end 5b of the fixation member 3a, 3b, so that the thickness of the rim 8 is a maximum at the second end 5b of the fixation member which comprises the projection 6.

Although FIG. 1 shows a rim 8 is provided on a first side 13a of each fixation member 3a, 3b, it will be understood that a second rim 8 could be provided on a second, opposite, side 13b of the each fixation member 3a, 3b. Each of the rims 8 which are provided on the second, opposite, side 13b of each fixation member 3a, 3b will also each comprise a surface 9 which, in the direction from a first end 5a of the fixation member 3a, 3b to a second end 5b of the fixation member 3a, 3b, veers towards the housing 2 of the valve device 1.

During use a removing tool comprising fixed elements, wherein the distance between the elements is equal to the maximum distance between said surfaces 9 of the rims 8, is positioned so that elements of the removing tool 10 abuts the surface 9 of a rim of a respective fixing element 3a, 3b. The removing tool 10 is advanced towards the valve device 1 so that the elements are slid along the surface 9 of a rim of a respective fixing element 3a, 3b, from the first end 5a of the fixing element to the second end 5b of the fixing element 3a, 3b. Since the elements of the removing tool 10 are fixed, and since the surface 9 of each rim 8 veers towards the housing 2 of the valve device 1, as the elements are slid along the surface 9 of a rim of a respective fixing element 3a, 3b they will push the fixing elements 3a, 3b apart so that the fixing elements will be flexed in a direction away from the housing 2 of the valve device 1. As the fixing elements 3a, 3b are flexed away from the housing 2, thus the fixing elements 3a, 3b can be ejected from corresponding recesses on the access port, so that a valve device 1 is no longer fixed to the access port. Additionally, when flexed in a direction away from the housing, the ledge 7 on each fixing element 3a, 3b will mechanically cooperate with members of a clip in the removing tool 10, so that the members of the clip hold the valve device 1 at the fixation members 3a, 3b by friction. Accordingly, the valve device 1 will be removed from the access port and will be held by the removing tool.

During use a mounting tool 20 may cooperate with the valve device 1 shown in FIG. 1 and may be used to mount the valve device 1 on an access port. As will be discussed in more detail later, the ledge 7 on each fixing element 3a, 3b can mechanically cooperate with members of a clip in the mounting tool 20, so that the members of the clip hold the valve device 1 at the fixation members 3a, 3b only when the fixation members 3a, 3b of the valve device 1 are flexed as the valve device 1 is being snap fitted to into position on the access port to detachably fix to the access port.

FIG. 2 shows a perspective view of a removing tool 10 according to a further aspect of the present invention. The removing tool 10 is suitable for removing the valve device 1 shown in FIG. 1 which is detachably fixed to an access port, from the access port. The removing tool 10 is defined by a single molded structure. The removing tool 10 is moulded from transparent material so that the removing tool 10 is see-through.

The removing tool 10 comprises two elements 11a, 11b each of which can be arranged to abut with said surface 9 of the rim 8 provided on a respective fixation member 3a, 3b of the valve device 1. The elements 11a, 11b are fixed in the removing tool 10 (preferably, the elements are immovable with respect to a body 14 of the removing tool 10) so that each of the elements 11a, 11b which abut with said surface 9 of the rim 8 provided on a respective fixation member 3a, 3b is moved along said surface 9 as the removing tool 10 is advanced towards the valve device 1. Since the elements 11a, 11b of the removing tool 10 are fixed, and since the surface 9 of each rim 8 veers towards the housing 2 of the valve device 1, as the elements 11a, 11b are slid along the surface 9 of a rim of a respective fixing element 3a, 3b they will push the fixing elements 3a, 3b apart so that the fixing elements 3a, 3b of the valve device 1 will be flexed in a direction away from the housing 2 of the valve device 1 so that the projection 6 on the fixation members 3a, 3b are moved out of respective recesses on the access port and the valve device 1 becomes detached from the access port.

Each element 11a, 11b is defined by a tapered projection which projects from a body 14 of the removing tool 10. Preferably the longer side 15 of each tapered projection can abut said surface 9 of the rim 8 on a respective fixation member 3a, 3b.

The two elements 11a, 11b are arranged opposite one another on the removing tool 10. The distance between outer surfaces 16 of the elements 11a, 11b is equal to the shortest distance between the surface 9 of the rim 8 provided on the first fixation members 3b and the surface 9 of the rim 8 provided on the second fixation member 3b, taking into account tolerance variations of the manufacturing process and of the materials used.

The removing tool 10 further comprises a handle portion 17 by which a user can hold the removing tool 10. The user holds the removing tool 10 at the handle portion 17 when advancing the removing tool 10 towards the valve device 1. The handle portion is provided with ridges 18 which can provide for improved gripping of the removing tool 10. Reinforcing members 19a, 19b attach each of the elements 11a, 11b respectively to the handle portion 17, so that the reinforcing members 19a, 19b and handle portion 17 can provide structural support to each of the elements 11a, 11b.

The removing tool 10 further comprises a fixation clip 12 arranged for holding the valve device 1 after it has become detached from the access port, so that the valve device is fixed to the removing tool 10 after the valve device 1 has been detached from the access port by the elements 11a, 11b. The fixation clip 12 is configured so that it can hold the valve device at the fixation members.

The fixation clip 12 comprises a first and second members 12a, 12b which are arranged opposite one another on the removing tool 10. Each of the first and second members 12a, 12b comprise a projection 31 which can mechanically cooperate with the ledge 7 provided on a respective fixation member 3a, 3b of the valve device 1, to hold the valve device 1. Each projection 31 may mechanically cooperate with a respective ledge 7. In a variation of the embodiment the projection may c-shaped; this will allow the fixation members which have an elongate projection provided on their respective ledges, to snap-fit to a respective first and second member 12a, 12b, so that the valve device 1 is held in the fixation clip 12.

The first and second members 12a, 12b each comprise a tapered member 32 and said projection 31 is provided at an end 33 of the tapered member. Each tapered member is attached at its widest end 34 to the body 14 of the removing tool 10, and the narrowest end 33 of the tapered member is a free end, and the projection 31 is located at the narrowest end 33 of the tapered member 32.

The distance between the first and second members 12a, 12b is such that moving the fixation members 3a, 3b of the valve device 1 apart using the elements 11a, 11b of the removing tool 10, by advancing the removing tool 10 towards the valve device 1, moves a first fixation member 3a so that the projection 31 on the first member 12a mechanically cooperates with the ledge 7 provided on the first fixation member 3a, and, moves the second fixation member 3b so that the projection 31 on the second member 12b mechanically cooperates with a ledge 7 provided on the second fixation member 3b, so that the fixation clip 12 holds the valve device 1 at its fixation members 3a, 3b. The projection 31 on each of the members 12a, 12b mechanically cooperates with a respective ledge 7. Thus the fixation clip 12 is positioned and configured so that moving the fixation members 3a, 3b of the valve device apart, using the elements 11a, 11b of the removing tool 10, by advancing the removing tool towards the valve device 1, moves the fixation members 3a, 3b to clip into the fixation clip 12 of the removing tool 10, so that the fixation clip 12 holds the valve device 12 at its fixation members 3a, 3b.

The removing tool 10 further comprises at least two projecting platforms 35a, 35b which can frictionally engage a valve device 1. Each of the projecting platforms 35a, 35b further comprise nodules 36 which increase friction between a respective projecting platform and a valve device. The valve device 1 can also comprise notches 37 configured to cooperate with the nodules 36 to further increase friction between a respective projecting platform and a valve device 1. The projecting platforms 35a, 35b and nodules 36 will ensure that the valve device 1 is fixed more securely to the removing tool 10. In particular the projecting platforms 35a, 35b and nodules 36 can hold the valve device 1 in the removing tool 10 before the valve device 1 has been detached from the access port. Additionally the projecting platforms 35a, 35b will facilitate positioning of a valve device 1 in a predetermined position in the removing tool 10 when the valve device 1 is being removed from the access port.

During use, the removing tool 10 is arranged so that each of the two elements 11a, 11b abuts said surface 9 of the rim 8 of a respective fixation member 3a, 3b, at the first end 5a of each of said fixation member 3a, 3b. The user holds the removing tool 10 at the handle portion 17 and advances the removing tool 10 towards the valve device 1, detachably fixed to an access port. Each of the projecting platforms 35a, 35b, possibly comprising nodules 36, frictionally engages with the housing 2, possible comprising the notches 37 frictionally cooperating with the nodules 36, of the valve device 1. The projecting platforms 35a, 35b can further facilitate correct positioning of a valve device 1 in the removing tool 10. As the removing tool 10 is further advanced towards the valve device 1 each of the elements 11a, 11b slide along the respective surface 9 of the rim 8 which they abut; since said surface 9 of each rim 8 veers towards the housing 2 of the valve device 1, and since the elements 11a, 11b are fixed to the removing tool, as the removing tool 10 is advanced towards the valve device 1 the fixation members 3a, 3b on the valve device will be moved apart by the elements 11a, 11b. As the fixation members 3a, 3b are moved apart the projections 6 on each of the fixation members 3a, 3b will be removed from their respective recesses in the access port so that the valve device 1 is no longer detachably fixed to the access port.

Once the fixation members 3a, 3b on the valve device are moved apart by the elements 11a, 11b on the removing tool 10, the projections 31 on each of the first and second members 12a, 12b of the fixation clip 12 will mechanically cooperate with the ledge 7 on a respective fixation member 3a, 3b of the valve device 1, to hold the valve device 1 at the fixation members 3a, 3b. Thus, the valve device 1 is detached from the access port by the elements 11a, 11b by advancing the removing tool 10 towards the valve device 1, and is then held by the first and second members 12a, 12b of the fixation clip 12 of the removing tool 10 after the valve device 1 has been detached from the access port.

Once the valve device 10 is held at the fixation members 3a, 3b by the first and second members 12a, 12b of the fixation clip 12, the valve device 1 will be fixed to the removing tool 10. Typically the user will dispose of the removing tool 10 and valve device 1 once the valve device 1 is fixed to the removing tool 10.

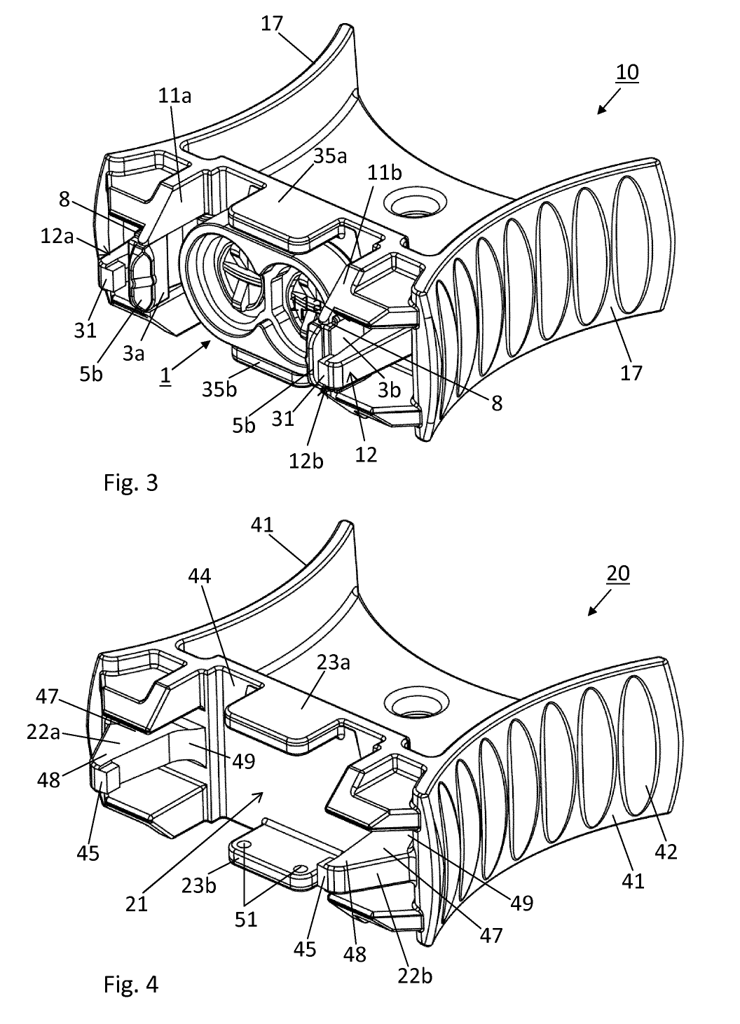

FIG. 3 illustrates an assembly according to an aspect of the present invention. Specifically, FIG. 3 shows a perspective view of the removing tool 10 of FIG. 2 with the valve device 1 of FIG. 1 fixed to the removing tool 10 after having been detached from an access port. In FIG. 3 the removing tool 10 has been advanced towards the valve device 1 so that the each of the elements 11a, 11b have been slid along the whole length of respective surface 9 of a rim 8 on a respective fixation member 3a, 3b, from the first end 5a of the respective fixation member 3a, 3b to the second end 5b of the respective fixation member 3a, 3b. The removing tool 10 has been advanced towards the valve device 1 until each of the elements 11a, 11b has reached the thickest part of the rim 8 which is located the second end 5b of each respective fixation member 3a, 3b; this will ensure that the fixation members 3a, 3b have been moved apart by a maximum distance by the elements 11a, 11b. The fixation members 3a, 3b on the valve device 1 have been moved apart by the elements 11a, 11b so that the projections 31 on the first and second members 12a, 12b of the fixation clip 12 mechanically cooperate with the ledge 7 on a respective fixation member 3a, 3b of the valve device 1, to hold the valve device 1 at the fixation members 3a, 3b. The projecting platforms 35a, 35b and the nodules 36 which are provided on the projecting platforms 35a, 35b ensure that the valve device is held more securely in the removing tool 10; additionally the projecting platforms 35a, 35b facilitate correct positioning of a valve device 1 in the removing tool 10.

It should be understood that the removing tool 10 may comprise any suitable number of elements 11a, 11b. For example, in another variation of the embodiment shown in FIG. 2, the removing tool 10 may comprise two pairs of elements the elements of each pair been arranged opposite one another on the removing tool 10. The elements are preferably arranged such that the distance between outer surfaces of the elements of one pair is equal to the shortest distance between said surfaces of rims provided on one side of a pair of fixation members of the valve device 1, and the distance between outer surfaces elements of the other pair is equal to the shortest distance between said surfaces of rims provided on an opposite side of said pair of fixation members of the valve device 1. Thus, four elements would be provided; a first element which can be arranged to abut said surface of a rim provided at one side of a first fixation member, and, a second element which can be arranged to abut said surface of a rim provided at the opposite side of the first fixation member; and a third element which can be arranged to abut said surface of a rim provided at one side of a second fixation member, and, a fourth element which can be arranged to abut said surface of a rim provided at the opposite side of the second fixation member. The first and third element will define the first pair of elements and the second and fourth element will define the second pair of elements.

FIG. 4 shows a perspective view of a mounting tool 20 according to a further aspect of the present invention. The mounting tool 20 is suitable mounting the valve device 1 shown in FIG. 1 on to access port so that the valve device 1 is detachably fixed to the access port. The mounting tool 20 is defined by a single molded structure. The mounting tool 20 is moulded from transparent material so that the mounting tool 20 is see-through. Preferably, the mounting tool is composed of a colored material, the color possibly corresponding to a code such as “attention, not to be reused!”.

The mounting tool 20 comprises a handle portion 41 by which a user can hold the mounting tool 20. The handle portion 41 is further provided with ridges 42 which can provide for improved gripping of the mounting tool 20.

The mounting tool 20 further comprises a cavity 21 defined in a body 44 of the mounting tool 20, suitable for receiving the valve device 1 of FIG. 1 which is to be mounted on the access port.

The mounting tool further provided with a holding clip 22 into which the fixation members 3a, 3b of a valve device 1 which is positioned in the cavity 21 can clip, only when the fixation members 3a, 3b are flexed apart. Thus the holding clip 22 will hold the valve device in the cavity 21 only when the fixation members 3a, 3b are flexed apart. Preferably, the holding clip 22 is configured to release the fixation members 3a, 3b when fixation members 3a, 3b recoil from their flexed position.

The holding clip 22 comprises a first and second member 22a, 22b, each of which can grip a fixation member 3a, 3b of the valve device 1, so that the fixation members 3a, 3b are clipped into the holding clip 12. Specifically, each of the first and second members 22a, 22b comprises a projection 45 which can grip the ledge 7 provided on a respective fixation member 3a, 3b of the valve device 1. When the projections 45 on each of the first and second members 22a, 22b grip the ledge 7 provided on the fixation members 3a, 3b then the fixation members 3a, 3b considered to clipped into the holding clip 22.

Each of the first and second members 22a, 22b comprises a tapered member 47 and said projection 45 is provided at an end 48 of the tapered member. Each tapered member is attached at its widest end 49 to the body 44 of the removing tool 20, and the narrowest end 48 of each tapered member 47 is a free end, and the projection 45 is located at the narrowest end 48 of the tapered member 47.

As the valve device 1 snap fits to detachably fix to the access port, the fixation members 3a, 3b will be flexed apart as the valve device 1 as the projections 6 on the fixation members 3a, 3b slide over a retaining lip provided on the access port. The fixation members 3a, 3b recoil from their flexed position after the projections 6 on the fixation members 3a, 3b have slid over a retaining lip and are received into corresponding recesses on the access port to detachably fix the valve device 1 to the access port. The distance between the first and second members 22a, 22b of the holding clip 22 is such that, said projection 45 on the first member 22a can grip the ledge 7 provided on the first fixation member 3a only when the first fixation member 3a is flexed as the projection 6 on the first fixation member 3a is sliding over the retaining lip provided on the access port, and, said projection on the second member 22b can grip a ledge 7 provided on the second fixation member 3b only when the second fixation member 3b is flexed as the projection 6 on the second fixation member 3a is sliding over the retaining lip provided on the access port. This will ensure that the holding clip 22 holds the valve device 1 only when the fixation members 3a, 3b are flexed apart as the valve device 1 is being pushed into a position on an access port where it is to be detachably fixed.

The first and second members 22a, 22b of the holding clip 22 are configured such that the fixation members 3a, 3b of the valve device are released from the holding clip 22 when the fixation members recoil. This can be achieved by separating the first and second members 22a, 22b of the holding clip 22 by an appropriate distance. The fixation members 3a, 3b will recoil from their flexed position after the projections 6 on the fixation members 3a, 3b have slid over the retaining lip and are received into corresponding recesses on the access port, to detachably fix the valve device 1 to the access port. Thus once the valve device 1 has become detachably fixed to the access port it will be released from (i.e. unclipped from) the holding clip 22 of the mounting tool 20.

The mounting tool 20 further comprising two projecting platforms 23a, 23b which are positioned on opposite sides of the cavity 21, which are used to hold the valve device 1 in predetermined position in the cavity 21. Each projecting platform 23a, b is fixed to the body 44 of the mounting tool 20. Each of the projecting platforms 23a,b comprise nodules 51 which increase friction between a valve device 1 in the cavity 21 and each projecting platform 23a,b so that the valve device 1 is held more securely in the cavity 21. Again, the valve device 1 can comprise notches 37 configured to cooperate with the nodules 51 to further increase friction between a respective projecting platform 23a, 23b and a valve device 1.

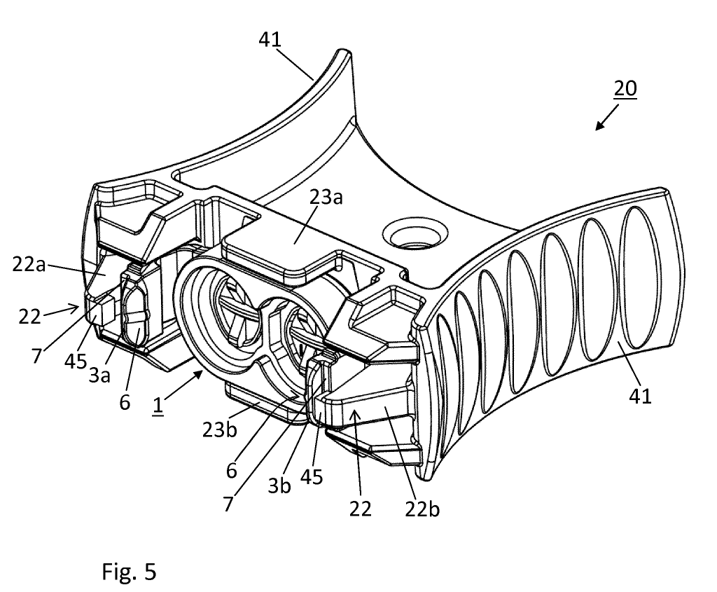

FIG. 5 illustrates an assembly according to a further aspect of the present invention. Specifically, FIG. 5 shows a perspective view of the mounting tool 20 of FIG. 4 with the valve device of FIG. 1 held in the cavity 21 of the mounting tool 20. Frictional force between the nodules 51 on each of the projecting platform 23a, 23b and the valve device 1 holds the valve device 1 in the cavity 21 of the mounting tool 20. As can been seen in FIG. 5 the fixation members 3a, 3b of the valve device 1 are not flexed and therefore the projections 45 on the first and second members 22a, 22b of the holding clip 22 do not grip the ledge 7 provided on a respective fixation member 3a, 3b of the valve device 1; accordingly as shown in FIG. 5 the valve device 1 is not clipped into the holding clip 22.

During use the valve device 1 which is to be mounted on an access port is positioned into the cavity 21 of the mounting tool 20 to form an assembly as shown in FIG. 5. The valve device 1 will be held in the cavity 21 by the modules 51 on each of the projecting platforms 23a, 23b.

With the valve device 1 held in the cavity 21 of the mounting tool 20, the mounting tool 20 is held by the user at the handle portion 41 and moved so that the valve device 1 is brought to the position on the access port where it is to be detachably fixed. The user pushes the valve device 1 into position on the access port using the mounting tool 20. The fixation members 3a, 3b on the valve device 1 are designed to snap-fit into the access port to detachably fix the valve device to the access port, thus the fixation members 3a, 3b will be flexed apart as the valve device 1 as the projections 6 on the fixation members 3a, 3b slide over a retaining lip provided on the access port. Once the fixation members 3a, 3b have been flexed apart they will clip into the holding clip 22; specifically the projection 45 provided on the first member 22a of the holding clip 22 will grip the ledge 7 provided the first fixation member 3a of the valve device 1 and the projection 45 provided on the second member 22b of the holding clip 22 will grip the ledge 7 provided the second fixation member 3b of the valve device 1, by friction so that the fixation members 3a, 3b are clipped into the holding clip 22. In this position the first and second members 22a, 22b of the holding clip 22 will prevent the valve device 1 from becoming removed from the cavity 21 of the mounting tool 20, until the fixation members 3a, 3b recoil again.

The fixation members 3a, 3b will recoil from their flexed position after the projections 6 on the fixation members 3a, 3b have slid over a retaining lip on the access port and are received into corresponding recesses on the access port, to detachably fix the valve device 1 to the access port (i.e. once the valve device has been successfully snap fitted into its position on the access port). Since the recoil force of the fixation members 3a, 3b is larger than the frictional force between the projections 45 on the first and second members 22a, 22b of the holding clip 22 and the respective ledges 7 of the first and second fixation members 3a, 3b, the first and second fixation members 3a,b will be released from the holding clip 22 once the fixation members 3a, 3b have been slid over a retaining lip on the access port and have been received into corresponding recesses on the access port. Thus the first and second fixation members 3a, 3b will be released from the holding clip 22 once the valve device 1 has been moved into the correct position on the access port where it becomes detachably fixed to the access port. Accordingly the holding clip 22 will hold the valve device 1 in the cavity 21 of the mounting tool 20 as valve device 1 is being pushed into position on the access port; and once the valve device 1 has been pushed into position on the access port where it is detachably fixed to the access port, the valve device 1 will be released from the holding clip 22.

With the valve device 1 detachably fixed to the access port the user may then gip the handle portion 41 of the mounting tool 20 and pull the mounting tool 20 from the valve device 1. The projections 6 on the fixation members 3a, 3b which have been received into respective recesses on the access port, will prevent the valve device 1 from moving with the mounting tool 20 as it is pulled away from the valve device 1. The pulling force applied by the user should be sufficient to overcome the frictional force which exists between the nodules 51 on the projections 23a, 23b of the valve device 1 so that the mounting tool 20 can be separated from the valve device 1. Thus the mounting tool 20 can be removed leaving the valve device 1 detachably fixed to the access port. The mounting tool 20 can be reused to mount another valve devicel to the/another access port.

Various modifications and variations to the described embodiments of the invention will be apparent to those skilled in the art without departing from the scope of the invention as defined in the appended claims. Although the invention has been described in connection with specific preferred embodiments, it should be understood that the invention as claimed should not be unduly limited to such specific embodiment.

### REFERENCE NUMBERS

1 valve device
2 housing
3 fixation clip
3a, 3b fixation member
4a, 4b valves
5a first end of the fixation member
5b second end of the fixation member
6 projection
7 ledge
8 rims
9 surface of the rims
10 removing tool
11a, 11b elements
12 fixation clip
12a first member of fixation clip
12b second member of fixation clip
13a first side
13b second side
4 body
15 longer side of each tapered projection
16 outer surfaces of the elements
17 handle portion
18 ridges
19a, 19b reinforcing members
20 mounting tool
21 cavity
22 holding clip
22a first member of holding clip
22b second member of holding clip
23a, 23b projecting platforms
31 projection
32 tapered member
33 narrowest end of tapered member
34 widest end of tapered member
35a, 35b projecting platforms
36 nodules
37 notches
41 handle portion
42 ridges
44 body of mounting tool
45 projection
47 tapered member
48 narrowest of tapered member
49 widest end of tapered member
51 nodules

1. A valve device suitable for detachably fixing to an access port, the valve device comprising,
one or more valves which are selectively operable to be in an open or closed configuration;
a fixation clip which comprises at least a first and second fixation member, wherein, the fixation members are configured to mechanically cooperate with the access port to detachably fix the valve device to the access port,
wherein each of the fixation members comprise one or more rims which project from a surface of a respective fixation member and which extend along at least a portion of the length of the respective fixation member, and wherein each of said one or more rims have a surface which veers in a direction towards the valve device, so that a tool which mechanically cooperates with said surfaces of the rims can be moved in a direction towards the valve device to cause the fixation members to be flexed apart to remove the valve device from the access port.

2. A valve device according to claim 1, wherein the valve device comprises a housing which houses the one or more valves, and wherein each fixation member has a first end which is secured to the housing and a second, free end, and,
wherein each rim is configured so that said surface of the rim veers in a direction towards the housing, in the direction from the first end of the fixation member to the second end of the fixation member.

3. A valve device according to claim 2, wherein each of the fixation members comprises a projection which is located at the second, free end of the fixation member, which can be received into a recess provided on the access port to detachably fix the valve device to the access port.

4. A valve device according to claim 3, wherein the rim on each fixation member is configured so that said surface is substantially perpendicular to the direction in which the projection projects from that fixation member.

5. A valve device according to claim 3, wherein the thickness of each rim provided on a fixation member increases along the fixation member, and wherein the thickness of the rim is a maximum at the second, free end of the fixation member.

6. A valve device according to claim 2, wherein each fixation member comprises flexible material which allows the fixation member to flex in a direction away from the housing.

7. A valve device according to claim 1, wherein each fixation member comprises a ledge which can mechanically cooperate with a clip of a tool so that the tool can hold the valve device at the fixation members.

8. A removing tool for removing a valve device which comprises one or more valves which are selectively operable to be in an open or closed configuration and a fixation clip which comprises at least a first and second fixation member, wherein the fixation members are configured to mechanically cooperate with the access port to detachably fix the valve device to the access port, each of the fixation members comprising one or more rims each of which having a surface which veers in a direction towards the valve device, so that a tool can mechanically cooperate with said surfaces of the rims to remove the valve device from the access port;
said valve device being detachably fixed to an access port from the access port, the removing tool comprising:
at least two elements each of which can be arranged to abut with said surface of the rim provided on the respective fixation member of the valve device, wherein the elements are fixed in the removing tool so that each of the elements is moved along said surface of the rim of the respective fixation member as the removing tool is advanced towards the valve device, so that the fixation members are moved apart and the valve device becomes detached from the access port; and
a fixation clip arranged for holding the valve device after it has become detached from the access port, so that the valve device is fixed to the removing tool after the valve device has been detached from the access port by the elements.

9. A removing tool according to claim 8, wherein the fixation clip is positioned so that moving the fixation members apart using the elements by advancing the removing tool towards the valve device, moves the fixation members to clip into the fixation clip, so that the fixation clip holds the valve device at its fixation members.

10. A removing tool according to claim 8, wherein the elements are arranged opposite one another and wherein the distance between outer surfaces of the elements is equal to the shortest distance between the surfaces of rims provided on said fixation members of the valve device.

11. A removing tool according to claim 8, wherein the fixation clip comprises a first and second member which are arranged opposite one another, and wherein each of the first and second member comprises a projection which can mechanically cooperate with a ledge provided on a respective fixation member of the valve device, to hold the valve device.

12. A removing tool according to claim 11, wherein the distance between the first and second members is such that moving the fixation members of the valve device apart using the elements by advancing the removing tool towards the valve device, moves a first fixation member so that the projection on the first member mechanically cooperates with a ledge provided on the first fixation member, and, moves the second fixation member so that the projection on the first member mechanically cooperates with a ledge provided on the second fixation member, so that the fixation clip holds the valve device at its fixation members.

13. A removing tool according to claim 8, wherein the removing tool further comprises at least two projecting platforms which can frictionally engage a valve device which is held by the fixation clip.

14. A mounting tool for mounting a valve device in an access port, the valve device comprising, one or more valves which are selectively operable to be in an open or closed configuration and a fixation clip which comprises at least a first and second fixation member, wherein the fixation members are configured to mechanically cooperate with the access port to detachably fix the valve device to the access port, each of the fixation members comprising one or more rims each of which having a surface which veers in a direction towards the valve device, so that a tool can mechanically cooperate with said surfaces of the rims to remove the valve device from the access port; the mounting tool comprising:
a cavity defined in a body of the mounting tool, suitable for receiving a valve device; and
a holding clip into which the fixation members of a valve device which is positioned in the cavity can clip only when the fixation members are flexed apart, so as to hold the valve device in the cavity only when the fixation members are flexed apart.

15. A mounting tool according to claim 14, wherein the holding clip comprises a first and second member, and wherein each of the first and second members comprises a projection which can grip a ledge provided on a respective fixation member of the valve device, by friction, only when the fixation members are flexed apart, so that the fixation members are clipped into the holding clip.

16. A mounting tool according to claim 15, wherein the distance between the first and second members is such that the fixation members clip into the holding clip only when the fixation members are flexed apart.

17. A mounting tool according to claim 15, wherein the first and second members are configured such that frictional force between a projection and respective ledge is less than the recoil force of the flexed fixation member, so that when the valve device has been moved into position in the access port where it is to be detachably fixed, the fixation members will retract from their flexed position to release the valve device from the holding clip.

18. A mounting tool according to claim 14, further comprising one or more projecting platforms which hold a valve device in the cavity of the mounting tool by friction.

19. A mounting tool according to claim 14, wherein the mounting tool comprises transparent material.

20. An assembly comprising
a valve device, which comprises one or more valves which are selectively operable to be in an open or closed configuration: a fixation clip which comprises at least a first and second fixation member, wherein the fixation members are configured to mechanically cooperate with the access port to detachably fix the valve device to the access port, each of the fixation members comprising one or more rims each of which having a surface which veers in a direction towards the valve device, so that a tool can mechanically cooperate with said surfaces of the rims to remove the valve device from the access port,
and a mounting tool comprising a cavity defined in a body of the mounting tool, suitable for receiving the valve device; and a holding clip into which the fixation members of the valve device which is positioned in the cavity can clip only when the fixation members are flexed apart, so as to hold the valve device in the cavity only when the fixation members are flexed apart;
wherein the valve device is located in the cavity of the mounting tool.
